(19) United States
(12) Patent Application Publication
(10) Pub. No.: US 2006/0222612 A1
(43) Pub. Date: Oct. 5, 2006

(54) Compositions of zirconium chloride complex and its method of manufacture

(75) Inventors: Hangsheng Ni, Haimen City (CN); Zhenjia Xu, Haimen City (CN)

Correspondence Address: Raymond Y. Chan, 108 N. Ynez Ave., #128, Monterey Park, CA, 91754, US

(73) Assignee: Haimen City Payuan Chemical Co., Ltd.

(21) Appl. No.: 11/099,199

(22) Filed: Apr. 4, 2005

(51) Int. Cl. A61K 8/58; A61K 8/28; C07F 7/28

(52) U.S. Cl. 424/66; 556/55

(57) ABSTRACT

The invention discloses a process for preparing highly active aluminum zirconium tetrochlorohydrate glycine powder by forming aluminum chloride glycine and zirconium chloride glycine solution individually, and then blending such aluminum chloride glycine and zirconium chloride glycine together to form aluminum zirconium tetrochlorohydrate glycine.

### BACKGROUND OF THE PRESENT INVENTION

1. Field of Invention

This invention relates to the production of antiperspirant compositions, and more particularly, relates to an improved process for preparing aluminium chloride hexahydrate, zirconium chloride, as well as aluminum zirconium tetrochlorohydrate (ZACH) complexed with a buffering agent glycine.

2. Description of Related Arts

The deodorants and antiperspirants market around the world is estimated to make up a third of the entire cosmetics market. Sweating plays a vital role in adjusting the body's temperature, that is to say the evaporation of a watery fluid from sweat glands on the surface of skin has a cooling effect.

Sweat glands, of which there are two types, occur over most of the body surface, but most abundantly in the armpit, the groin, on the soles of the feet and the palms of the hands, and on the forehead. They produce a dilute aqueous solution containing mainly NaCl and urea, but also other metabolic waste products, such as lactates produced in muscles, and sparse amounts of protein- and lipid-rich secretions, which include cholesterol and steroids. These fatty compounds are broken down by bacteria on the skin surface, mostly to low carbon chain (C₄-C₁₀) fatty acids such as 3-methyl-2-hexanoic acid, which we recognize as body odour (BO).

In short, the sweat perspiration will provide odor and wetness. It is witnessed that substantial attempts have been devoted in this market as Antiperspirants containing aluminum salts, which physically block the sweat glands. Meanwhile, aluminum salts are also antibacterial agents and therefore have a deodorizing effect.

It is well known within the art that aluminum salt dissolves into the neck of the sweat duct. The average pH of underarm sweat is six, so the aluminum salts form polymeric aluminum hydroxide solids formed as a gel, which blocks the sweat gland duct thus reducing the amount of sweat secreted.

The first branded antiperspirant launched in 1902. It was an aqueous solution of AlCl₃ which was dabbed onto the armpits with cotton wool. Unfortunately with a pH of two, the antiperspirant was so acidic that it irritated the skin and rotted clothes.

Generally, the most basic aluminum salts used a general formula Al₂ (OH)ₘXₙ, where m+n=6, in antiperspirant formulations. Aluminium chlorohydrate (ACH), where X=Cl, is still commonly used in antiperspirants today. Later on, zirconium salts are introduced in antiperspirants market. The zirconium salts have similar chemistry to aluminum salts in forming gels on hydrolysis, but have slightly more efficient than zluminium salts because of the higher acidity and greater coordinating power of Zr⁴⁺ containing complexes.

However, the zirconium are too expensive to use as the sole antiperspirant active but incorporating a small amount of zirconium oxydichlorohydrate (ZrOCl₂) or zirconium oxyhydroxychlorohydrate (ZrO(OH)Cl) in ACH-based antiperspirants improves their efficacy by 30-50 percent. These zirconium/aluminium chlorohydrate (ZACH) salts are more acidic than ACH and therefore need to be buffered to reduce skin irritation. This led to the development of ‘ZAG salts’: ZACH salts complexed with glycine (aminoethanoic acid), which buffers ZACH salts without hindering performance.

Alough the ACH and ZACH have been known for many years to be an effective and safe antiperspirant. Nevertheless, there is room for improvement of the activity of the final products.

### SUMMARY OF THE PRESENT INVENTION

A primary object of the present invention is to provide highly active and effective antiperspirant compositions, including ACH and ZACH salts.

Another object of the present invention is to provide ACH and ZACH salts having an efficient HPLC analysis result.

Accordingly, the present invention provides a process for preparing aluminum/zirconium tetro-chlorohydrate glycine, comprising:

(a) Forming an aluminum chlorohydrate solution;

(b) Adding glycine to the aluminum chlorohydrate solution to from aluminum-glycine solution;

(c) Forming a zirconium chlorohydrate solution;

(d) Adding glycine to the zirconium chlorohydrate solution to form zirconium-glycine solution; and

(e) Blending the aluminum-glycine solution with the zirconium-glycine solution to form aluminum/zirconium tetro-chlorohydrate glycine solution; and

(f) spray-drying the aluminum/zirconium tetro-chlorohydrate glycine solution to form powder of the aluminum/zirconium tetro-chlorohydrate glycine.

These and other objectives, features, and advantages of the present invention will become apparent from the following detailed description, the accompanying drawings, and the appended claims.

### DETAILED DESCRIPTION OF THE PREFERRED EMBODIMENT

The present invention introduces a method for preparing aqueous solution and powder of aluminum/zirconium tetrochlorohydrate glycine. The method comprises following steps:

(a) forming an aluminum chlorohydrate solution;

(b) adding glycine to the aluminum chlorohydrate solution to from aluminum-glycine solution;

(c) forming a zirconium chlorohydrate solution;

(d) adding glycine to the zirconium chlorohydrate solution to form zirconium-glycine solution; and

(e) blending the aluminum-glycine solution with the zirconium-glycine solution to form aluminum/zirconium tetro-chlorohydrate glycine solution; and

(f) spray-drying the aluminum/zirconium tetro-chlorohydrate glycine solution to form powder of the aluminum/zirconium tetro-chlorohydrate glycine.

In the step (a), 1.56 kg Al(OH)₃ is added into a 5 L round bottom flask with a reflux condenser, then 2.45 kg hydrochloric acid of 20 Baume degree is slowly dripped into the round bottom flask, followed by a stirring process of 1-12 hours at a temperature between 30-70° C., to form aluminum chloride hexahydrate. The analysis shows the Al content is 18.18% and the Cl content is 52.51%.

The step (a) further comprises a step (a-1) for adding 0.75 kg of de-ionized water and 0.95 kg aluminum into the aluminum chloride hexahydrate, and a step for heating up the solution to a temperature with 95±3° C. During the step, there are a plenty of air bubbles generated at an earlier stage of the reaction. After the reaction is continuously reacted 54-78 hours, the aluminum chloroide hydrate is obtained. A further analysis shows the Al content is 9.75%, the Cl content is 7.14%, and the atom ratio of Al/Cl is 1.80.

Afterwards, 4.53 kg de-ionized water is added into the aluminum chloride hydrate to dilute the solution. Under a pressure of 0-5 kg f/cm², the reaction temperature is risen to a temperature ranging from 98° C. to 158° C., and the heating process will be maintained at a period from 0.5 hour to 5 hours so as to form highly active aluminum chloride hydrate.

According to the present invention, the aluminum chloride hydrate has a chemical formula Al₂ (OH)_{(6-X)}X_{X}, where x is varying from 0.3 to 6.

In the step (b), 0.01 kg glycine is added into the aluminum chloride hydrate, after a 1-5 hours stirring process under a constant temperature ranging from 50° C. to 90° C., highly active aluminum chloride glycine is formed.

In the step (c), 0.87 kg basic zirconium carbonate is added into a 5 L round bottom flask with a reflux condenser, and then 0.41 kg of hydrochloric of 20 Baume degrees is slowly dripped into the round bottom flask. In this stage, along the adding of the hydrochloric acid, the reaction is acute, creating a plenty of air bubble. Afterwards, a 0.2-2.5 hour stirring process is followed, and then the solution is heat to a temperature ranging from 90° C. to 98° C., and is refluxed 0.5 to 5 hours to form basic zirconium chloride.

In the step (d), 0.15 kg of glycine is added into the basic zirconium chloride, and followed by a stirring process ranging 0.2-5 hours to form basic zirconium chloride glycine.

In the step (e), the basic zirconium chloride glycine prepared by step (d) is slowly added into the aluminum chloride glycine prepared by step (b), afterwards, the mixture is stirred up 0.5 to 5 hours under a constant temperature ranging from 50-90° C. to form highly active aluminum-zirconium tetrachlorohydrate solution.

In the step (f), after a brief cooling process, the highly active aluminum-zirconium tetrachlorohydrate solution is centrifuged within spraying dryer, wherein the hot air inlet of the spraying dryer is set to a temperature 105±10° C. to obtain highly active aluminum-zirconium tetrachlorohydrate solution powder.

The final powder is analyzed by a HPLC chromatography, showing the peak area 1 and peak area 2 are less than 1% of total area, and the area ration between peak 4 and peak 3 is around 1.20.

It is noted that the aluminum zirconium tetrochlorohydrate glycine has a chemical formula Alₓ(OH)_{y}ZrOCl₃ₓ₊₂;Gly, wherein x is varying from 0-2, and y is varying from 2.7 to 5.3.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A process for preparing aluminum/zirconium tetro-chlorohydrate glycine, comprising:
(a) forming an aluminum chlorohydrate solution;
(b) adding glycine to said aluminum chlorohydrate solution to from an aluminum-glycine solution;
(c) forming a zirconium chlorohydrate solution;
(d) adding glycine to said zirconium chlorohydrate solution to form a zirconium-glycine solution;
(e) blending said aluminum-glycine solution with said zirconium-glycine solution to form aluminum/zirconium tetrochlorohydrate glycine solution; and
(f) spray-drying said aluminum/zirconium tetro-chlorohydrate glycine solution to form powder of said aluminum/zirconium tetro-chlorohydrate glycine.

2. The process, as recited in claim 1, wherein the step (a) further comprises following steps:
(a-1) treating Al(OH)₃ with hydrochloric acid of 20 Baume degree to form an aluminum chloride hexahydrate;
(a-2) adding de-ionized water and aluminum into said aluminum chloride hexahydrate;
(a-3) continuously heating up said aluminum chloride hexahydrate to a temperature with 95±3° C. with a predetermined period to form a preliminary aluminum chlorohydrate solution;
(a-4) diluting said preliminary aluminum chlorohydrate solution with de-ionized water,
(a-5) heating up said diluted preliminary aluminum chlorohydrate under a predetermined pressure and a predetermined temperature to form a highly active aluminum chlorohydrate.

3. The process, as recited in claim 2, wherein said predetermined period is within a range from 54-78 hours.

4. The process, as recited in claim 2, wherein said predetermined temperature is within a range from 50-90° C.

5. The process, as recited in claim 3, wherein said predetermined temperature is within a range from 50-90° C.

6. The process, as recited in claim 1, wherein the step (c) further comprises following steps:
(c-1) treating basic zirconium carbonate with hydrochloric acid of 20 Baume degree to form an zirconium chloride;
(c-2) stirring up said zirconium chloride at a predetermined time;
(c-3) heating up said zirconium chloride to a temperature ranging from 90° C. to 98° C.; and
(c-4) refluxing said zirconium chloride with a predetermined time.

7. The process, as recited in claim 2, wherein the step (c) further comprises following steps:
(c-1) treating basic zirconium carbonate with hydrochloric acid of 20 Baume degree to form an zirconium chloride;
(c-2) stirring up said zirconium chloride at a predetermined time;
(c-3) heating up said zirconium chloride to a temperature ranging from 90° C. to 98° C.; and
(c-4) refluxing said zirconium chloride with a predetermined time.

8. The process, as recited in claim 5, wherein the step (c) further comprises following steps:
(c-1) treating basic zirconium carbonate with hydrochloric acid of 20 Baume degree to form an zirconium chloride;
(c-2) stirring up said zirconium chloride at a predetermined time;
(c-3) heating up said zirconium chloride to a temperature ranging from 90° C. to 98° C.; and
(c-4) refluxing said zirconium chloride with a predetermined period of time.

10. The process, as recited in claim 6, wherein said predetermined time is within a range from 0.5 to 1.5 hour.

11. The process, as recited in claim 7, wherein said predetermined time is within a range from 0.5 to 1.5 hour.

12. The process, as recited in claim 8, wherein said predetermined time is within a range from 0.5 to 1.5 hour.

13. The process, as recited in claim 1, wherein said aluminum chlorohydrate solution has a chemical formula Al₂ (OH)_{(6-X)}X_{X}, where x is varying from 0.3 to 6.

14. The process, as recited in claim 2, wherein said aluminum chlorohydrate solution has a chemical formula Al₂ (OH)_{(6-X)}X_{X}, where x is varying from 0.3 to 6.

15. The process, as recited in claim 6, wherein said aluminum chlorohydrate solution has a chemical formula Al₂ (OH)_{(6-X)}X_{X}, where x is varying from 0.3 to 6.

16. The process, as recited in claim 7, wherein said aluminum chlorohydrate solution has a chemical formula Al₂ (OH)_{(6-X)}X_{X}, where x is varying from 0.3 to 6.

17. The process, as recited in claim 1, wherein said aluminum zirconium tetrochlorohydrate glycine has a chemical formula Alₓ(OH)_{y}ZrOCl3ₓ+2;Gly, wherein x is varying from 0-2, and y is varying from 2.7 to 5.3.

18. The process, as recited in claim 2, wherein said aluminum zirconium tetrochlorohydrate glycine has a chemical formula Alₓ(OH)_{y}ZrOCl3ₓ+2;Gly, wherein x is varying from 0-2, and y is varying from 2.7 to 5.3.

19. The process, as recited in claim 6, wherein said aluminum zirconium tetrochlorohydrate glycine has a chemical formula Alₓ(OH)_{y}ZrOCl3ₓ+2;Gly, wherein x is varying from 0-2, and y is varying from 2.7 to 5.3.

20. The process, as recited in claim 6, wherein said aluminum zirconium tetrochlorohydrate glycine has a chemical formula Alₓ(OH)_{y}ZrOCl3ₓ+2;Gly, wherein x is varying from 0-2, and y is varying from 2.7 to 5.3.
